(19) United States
(12) Patent Application Publication
(10) Pub. No.: US 2023/0021045 A1
(43) Pub. Date: Jan. 19, 2023

(54) A METHOD FOR CO-PRODUCING MONOPHENOLS AND CELLULOSE BY CATALYTIC OXIDATION OF BIOMASS OVER A TRANSITION METAL OXIDE

(71) Applicant: GUANGZHOU INSTITUTE OF ENERGY CONVERSION, CHINESE ACADEMY OF SCIENCES, Guangzhou City, Guangdong (CN)

(72) Inventors: Chenguang WANG, Guangzhou City, Guangdong (CN); Yuting ZHU, Guangzhou City, Guangdong (CN); Longlong MA, Guangzhou City, Guangdong (CN); Jing LIU, Guangzhou City, Guangdong (CN); Wei LV, Guangzhou City, Guangdong (CN); Qi ZHANG, Guangzhou City, Guangdong (CN)

(21) Appl. No.: 17/781,959

(22) PCT Filed: Jun. 15, 2020
(86) PCT No.: PCT/CN2020/096029

§ 371 Date: Jun. 2, 2022

(30) Foreign Application Priority Data
Dec. 3, 2019 (CN) 201911220057.1

(51) Int. Cl. C07C 37/54; D21C 5/00

(52) U.S. Cl. CPC C07C 37/54; D21C 5/00

(57) ABSTRACT

A method for co-production of monophenols and cellulose by transition metal oxide catalytic oxidation of biomass is disclosed. The method uses transition metal oxide as catalyst and pretreated dry biomass as raw material to obtain high purity and selectivity of monophenolic chemicals with co-produced cellulose under mild conditions.

### TECHNICAL FIELD

The present invention relates to the technical field of biomass-derived chemicals, in particular to a method for the co-production of monophenol compounds and cellulose by transition metal oxide catalytic oxidation of biomass.

### BACKGROUND

Researches on the substitution of lignocellulosic biomass for fossil resources to prepare chemicals and materials have attracted wide attention. Lignin, as one of the three main components of lignocellulosic biomass and rich in aromatic ring structures, is an ideal source for the preparation of monophenolic chemicals of high commercial value. However, due to its complex structure, it is difficult to be utilized efficiently. At present, main technologies for lignin conversion are pyrolysis, hydrogenation and oxidation, among which oxidation has the advantages of mild reaction conditions and high selectivity of monophenols.

The main products of lignin oxidation are aromatic aldehydes, i.e. vanillin, syringaldehyde and p-hydroxy benzaldehyde. These aromatic aldehydes have important applications in the fields of food, cosmetics, medicine, pesticide and chemical industry. Therefore, lignin oxidation technology has been widely studied. For instance, Praveen et al. used niobium oxalate as a catalyst to catalyze alkali lignin to obtain aromatic aldehyde in yield of 8.9% (vanillin, 6.6%; syringaldehyde, 2.3%) (L. Das, P. Kolar, J. A. Osborne, R. R. Sharma-Shivappa, J. J. Classen, Selective Oxidation of Lignin into Aromatic Aldehydes Using Niobium Oxalate. Transactions of the ASABE, 2016, 59, 727-735). Li et al. found porphyrin (Co(TPPS₄)) efficient in catalyze different biomass (including corn straw after enzymatic hydrolysis by steam explosion, lignocellulose residue rich in lignin after ethanol production) with the highest yield of aromatic aldehydes of 12.8% (Y. Li, J. Chang, Y. Ouyang. Selective Production of Aromatic Aldehydes from Lignin by Metalloporphyrins/H₂O₂ System. Advanced Materials Research, 2013, 805-806, 273-276). The substrates used in the above studies were extracted lignin or biomass pretreated under harsh conditions. The yield of monophenols was generally low because the lignin structure was heavily damaged during extraction or pretreatment, during which more stable condensation structure that is difficult to be depolymerized were formed. Moreover, the oxidant used in the above works was hydrogen peroxide which is not conducive to industrial production for its too strong oxidizing property.

The yield of monophenols can be significantly increased when raw biomass is used as raw material. For example, monophenols yield of 29.4% (vanillin selectivity, 71.8%) was obtained by oxidation of pine powder with oxygen as the oxidant in NaOH solution (Y. T. Zhu, J. Liu, Y. H. Liao, W. Lv, L. L. Ma, C. G. Wang. Degradation of Vanillin During Lignin Valorization Under Alkaline Oxidation. Topics in Current Chemistry, 2018, 376:29). However, the yield of monophenols obtained by the current oxidation process is lower than the theoretical yield, and the yield of cellulose is relatively low. The use of catalyst is expected for further improvement the yield of monophenols. Transition metal oxides such as CuO are commonly used catalysts for catalytic oxidation reactions. However, so far, the co-production of monophenols and high-purity cellulose from lignocellulosic biomass by catalytic oxidation of transition metal oxides is rarely reported.

In summary, using lignocellulosic biomass as raw material to prepare monophenolic chemicals and co-produce high-purity cellulose is an effective measure to deal with the current problems of shortage of fossil resources and environmental damage. However, existing oxidation technologies have problems such as unsafe oxidant, low product concentration and low yield of monophenols.

### SUMMARY

The present invention aims to overcome the drawbacks of existing technology, and to provide a green, safe, mild-condition, and simple method of transition metal oxide catalytic oxidation of biomass to phenolic compounds, which uses lignocellulose as raw material, oxygen as oxidant, transition metal oxides as catalyst; and co-produces monophenols and cellulose in moderate conditions, thereby the lignocellulose can be efficiently utilized.

A method for co-production of monophenol compounds and cellulose by transition metal oxide catalytic oxidation of biomass is provided comprising:

(a) a reactor is loaded with pretreated dry biomass, transition metal oxide and alkali metal hydroxide solution and then sealed, the reactor being evacuated and refilled with O₂, the O₂ pressure being kept at 0.1˜3 MPa, and the mixture is allowed to react at 80˜200° C. for 1˜180 min under stirring, and then cooled down at room temperature to obtain a suspension;

(b) the suspension obtained in step (a) is centrifuged to separate clear supernatant from solid residue, the supernatant being acidified to pH 2˜3, organic solvent being added to the acidified solution to extract phenolic compounds and the organic phase being gathered; and the residual acid and water in the organic solution are removed; and monophenols can be obtained by vacuum distillation of the organic phase; and

(c) the solid residue obtained in step (b) is placed in a sieve of 800 meshes, and the catalyst is rinsed out with water while the biomass residue retains on the sieve, the aqueous solution containing catalyst is filtered or centrifuged to recycle the spent catalyst, the biomass residue is washed with acid aqueous solution to remove the residual catalyst until the solution is clear, and continuously washed with water until the pH value of waste liquid reaches 7 and then the biomass residue is dried.

At step (b), strong base-weak acid salt is added to neutralize inorganic acid remaining in the organic solution, and the organic solution is dried by anhydrate Na₂SO₄. The organic solvent used in step (b) is preferably dichloromethane, chloroform, tetrahydrofuran, ethyl acetate or acetone. The obtained monophenols are mainly vanillin, syringaldehyde, p-hydroxy benzaldehyde.

The dry biomass is preferably softwood, hardwood, grass, or agro-forestry residue, and more preferably pine, eucalyptus, Chinese pennisetum, bagasse, or corn stalk. The lignin content of pine is about 23%, correspondingly, the theoretical maximum yield of monophenols of pine is 38%. The lignin content and theoretical maximum yield of monophenols of eucalyptus are about 26% and 50%, respectively.

Before the oxidation reaction, dry biomass is suffered to Soxhlet extraction to remove the ethanol-soluble oil and pigment. The typical steps of the soxhlet extraction are as follows: biomass is treated with anhydrous ethanol and refluxing for 8 h, then dried at 50˜60° C.

The hydroxide of the alkali metal is preferably NaOH, KOH, or Mg(OH)₂. The weight percent concentration of the aqueous solution of the hydroxide of the alkali metal is preferably at the range of 2.5˜15%.

Preferably, the solid-liquid ratio of dry biomass to aqueous solution of hydroxide of alkali metal is at the range of 1:20˜50.

Preferred transition metal oxides include CuO, TiO₂, NiO, Co₂O₃, Fe₂O₃, Fe₃O₄ and ZnO. The weight ratio of the transition metal oxide to the dry biomass is 1˜100%.

More preferably, the transition metal oxide is CuO nanoparticles with diameter of 40 to 200 nm, and the content of CuO nanoparticles is preferably 20˜40% of the weight of the dry biomass.

Preferred reaction conditions in step (a) are listed as follows: reaction temperature: 120˜180° C., agitation speed: 50˜1000 rpm, reaction time: 10˜120 min, oxygen pressure: 0.5˜1.5 MPa.

The present invention is advantageous and beneficial especially in that:

the raw materials used in the invention is of wide source, low cost and easy to harvest;

the catalysts used in the present invention are commercially available, which can be reused and easy to be industrialized; and

under mild conditions, lignin can be efficiently transformed into monophenols with aromatic aldehyde as the main component and co-producing high-purity cellulose, the yield of monophenols up to 46% and the selectivity of aromatic aldehyde up to 90%. Moreover, the yield and purity of cellulose are up to 90% and 98%, respectively.

### DETAILED DESCRIPTION OF THE INVENTION

The present invention as will be described in detail below should not to be understood to be limited to the particular methodologies.

The equipment and materials mentioned in the present invention are commercially available, unless otherwise stated. Unless otherwise specified, the percent sign “%” referred to in the present invention refers to the weight percentage.

The yield and selectivity of monophenol, yield and purity of cellulose are calculated by the following formula.

Yield of monophenol=(weight of monophenol/weight of lignin in pine)×100%

Selectivity of aromatic aldehyde=(weight of aromatic aldehyde/weight of all monophenols)×100%

Yield of cellulose=(weight of cellulose retained/weight of cellulose in pine)×100%

Selectivity of cellulose=(weight of cellulose retained/weight of biomass residue)×100%

### EXAMPLE 1

A method for co-production of monophenol compounds and cellulose by transition metal oxide catalytic oxidation of biomass is provided in this example. The method has the following steps.

(a) In a 50 mL autoclave reactor, 0.5 g of pretreated dry pine powder, 0.1 g (the weight of 20% of biomass) of 40 nm CuO, 25 mL of NaOH aqueous solution of 7.5%, were put into the reaction vessel and then sealed. The reactor was evacuated and refilled with O₂, the O₂ pressure was kept at 1 MPa. The reaction mixture was heated at 160° C. for 60 min under stirring speed of 400 rpm. After cooled down at room temperature, the reactor was open and a suspension was obtained.

(b) The suspension obtained in step (a) was centrifuged to separate clear supernatant and solid residue. The supernatant was acidified with hydrochloric acid (36% aqueous solution) until pH value was 2˜3. Dichloromethane was added to the acidified solution to extract phenolic compounds. The dichloromethane phase was gathered. NaHCO₃ was added into dichloromethane solution to neutralize residue hydrochloric acid, and then Na₂SO₄ was added to remove the remaining water. Finally, monophenols was obtained by vacuum distillation of the dichloromethane phase.

(c) The solid residue obtained in step (b) was placed in a sieve of 800 meshes, and the catalyst was rinsed out with water while the biomass residue retained on the sieve. The aqueous solution containing catalyst was filtered or centrifuged to obtain the spent catalyst, and the spent catalyst can be used in the next batch. The biomass residue was washed with hydrochloric acid aqueous solution to remove the residual catalyst until the solution clear, continue washed with water until the pH value of waste liquid reached 7. Finally, the biomass residue was dried.

The yield, selectivity and purity of products are listed in Table 1.

### EXAMPLES 2 TO 5

The preparation process is substantially the same as Example 1, except that the catalyst 40 nm CuO was replaced with 50 nm TiO₂, 20 nm Fe₂O₃, 50 nm ZnO and 20 nm Al₂O₃. The yield, selectivity and purity of products are listed in Table 1.

TABLE 1
Results of catalytic oxidation of pine over different metal oxides.

| | | Yield of | Selectivity | Yield of | Purity of |
|---|---|---|---|---|---|
| | | monophenols | of vanillin | cellulose | cellulose |
| Examples | Catalyst | (%) | (%) | (%) | (%) |
| Example 1 | 40 nm CuO | 36 | 73 | 80 | 95 |
| Example 2 | 50 nm TiO₂ | 22 | 70 | 48 | 95 |
| Example 3 | 20 nm | 17 | 72 | 30 | 92 |
| Example 4 | 50 nm ZnO | 18 | 71 | 35 | 98 |
| Example 5 | 20 nm | 21 | 70 | 50 | 96 |

It can be seen in Table 1 that all the selected transition metal oxides can catalyze the oxidation of biomass to monophenols and retain a portion of cellulose. Compared with other metal oxides, the nano CuO exhibits superior performance in catalytic oxidation of pine to produce monophenols and co-production of high-purity cellulose.

### EXAMPLES 6 TO 8

The preparation process is substantially the same as Example 1, except that the catalyst 40 nm CuO was replaced with 200 nm CuO, 10 μm CuO and 75 μm CuO. The yield, selectivity and purity of products are listed in Table 2.

TABLE 2
Results of catalytic oxidation of pine over CuO with different particle sizes.

| | | Yield of | Selectivity | Yield of | Purity of |
|---|---|---|---|---|---|
| | | monophenols | of vanillin | cellulose | cellulose |
| Examples | Catalyst | (%) | (%) | (%) | (%) |
| Example l | 40 nm CuO | 36 | 73 | 80 | 95 |
| Example 6 | 200 nm | 32 | 75 | 75 | 95 |
| Example 7 | 10 μm | 26 | 72 | 62 | 95 |
| Example 8 | 75 μm | 25 | 70 | 60 | 94 |

It can be seen in Table 2 that the yield of monophenols and cellulose increases gradually as the particle size of CuO particles decreases, and the 40 nm CuO shows the best catalytic activity.

### EXAMPLES 9 TO 12

The preparation process is substantially the same as Example 1, except that the dosage of 40 nm CuO was changed to 10%, 40%, 60% and 80% of the dosage of biomass. The yield, selectivity and purity of the products are shown in Table 3.

TABLE 3
Results of catalytic oxidation of pine over 40 nm
CuO with different dosage.

| | | | | | Purity |
|---|---|---|---|---|---|
| | Dosage 40 | Yield of | Selectivity | Yield of | of |
| | nm CuO | monophenols | of vanillin | cellulose | cellulose |
| Examples | (%) | (%) | (%) | (%) | (%) |
| Example 1 | 20 | 36 | 73 | 80 | 95 |
| Example 9 | 10 | 31 | 76 | 65 | 98 |
| Example 10 | 40 | 25 | 73 | 68 | 97 |
| Example 11 | 60 | 22 | 74 | 68 | 92 |
| Example 12 | 80 | 18 | 75 | 65 | 93 |

As can be seen in Table 3, the yields of monophenols and cellulose increases and then decreases with the increase of the dosage of 40 nm CuO, and the best catalytic performance is achieved at the dosage of 20%.

### EXAMPLES 13 TO 14

The preparation process is substantially the same as Example 1, except that the catalyst 40 nm CuO was replaced by catalyst reused at the second time and third time. The yield, selectivity and purity of the products are shown in Table 4.

TABLE 4
Results of catalytic oxidation of pine over 40 nm CuO.

| | | Yield of | Selectivity | Yield of | Purity of |
|---|---|---|---|---|---|
| | 40 nm CuO | monophenols | of vanillin | cellulose | cellulose |
| Example | used time | (%) | (%) | (%) | (%) |
| Example 1 | First time | 36 | 73 | 80 | 95 |
| Example 13 | Second time | 30 | 76 | 78 | 98 |
| Example 14 | Third time | 29 | 77 | 76 | 97 |

As can be seen in Table 4, nano CuO shows no obvious reduction of catalytic activity after three recycles, indicating its high reusability.

### EXAMPLES 15 TO 19

The preparation process is substantially the same as Example 1, except that the biomass pine was replaced by eucalyptus, Chinese pennisetum, bagasse, corn stalk, furfural residue of corn stalk. The yield, selectivity and purity of the products are shown in Table 5.

TABLE 5
Results of catalytic oxidation of different biomass over 40 nm CuO.

| | | | | | Selectivity | | |
|---|---|---|---|---|---|---|---|
| | | Yield | Selectivity | Selectivity | of | Yield | Purity |
| | | of | of | of | p-hydroxy | of | of |
| | | monophenols | vanillin | syringaldehyde | benzaldehyde | cellulose | cellulose |
| Examples | Biomass | (%) | (%) | (%) | (%) | (%) | (%) |
| Example 1 | pine | 36 | 73 | 0 | 0 | 80 | 95 |
| Examp [symbol] | eucalypt [symbol] | 46 | 24 | 66 | 0 | 80 | 98 |
| Examp [symbol] | Chinese [symbol] | 25 | 40 | 25 | 11 | 90 | 97 |
| Examp [symbol] | bagasse | 20 | 37 | 25 | 15 | 40 | 96 |
| Example 18 | corn stalk | 15 | 50 | 20 | 15 | 40 | 95 |
| Example 19 | furfural | 10 | 30 | 20 | 12 | 10 | 92 |
| | residue | | | | | | |

[symbol]

indicates data missing or illegible when filed

As can be seen in Table 5, nano CuO is efficient in catalyze oxidation of various biomasses to monophenols with aromatic aldehydes as main product and co-producing high-purity cellulose. Among the biomasses, the highest yield of monophnol achieves when eucalyptus is used as raw material, while Chinese pennisetum gives the highest yield of cellulose.

### EXAMPLE 20

The preparation process is substantially the same as Example 1, except that step (a) was modified as follows: In a 50 mL autoclave reactor, 0.5 g of pretreated dry pine powder, 0.2 g (the weight of 40% of biomass) of 40 nm CuO, 25 mL of NaOH aqueous solution of 2.5%, were put into the reaction vessel and then sealed. The reactor was evacuated and refilled with O₂, the O₂ pressure was kept at 0.5 MPa. The reaction mixture was heated at 120° C. for 120 min under stirring speed of 50 rpm. After cooled down at room temperature, the reactor was open and a suspension was obtained. The yield of monophenols was 15%, selectivity of vanillin was 72%; and the yield and purity of cellulose was 85% and 81%, respectively.

### EXAMPLE 21

The preparation process is substantially the same as the example 1, except that step (a) was modified as follows: In a 50 mL autoclave reactor, 0.5 g of pretreated dry pine powder, 0.1 g (the weight of 20% of biomass) of 40 nm CuO, 10 mL of NaOH aqueous solution of 15%, were put into the reaction vessel and then sealed. The reactor was evacuated and refilled with O₂, the O₂ pressure was kept at 1.5 MPa. The reaction mixture was heated at 180° C. for 10 min under stirring speed of 1000 rpm. After cooled down at room temperature, the reactor was open and a suspension was obtained. The yield of monophenols was 25%, selectivity of vanillin was 70%; and the yield and purity of cellulose was 60% and 74%, respectively.

While the specification has described in detail in certain exemplary examples, the description of the above examples is intended only to facilitate the understanding of the technical protocol of the invention and its spirit. It will be appreciated that those skilled in the art, upon attaining an understanding of the foregoing, may readily conceive of alterations to, modification of, and equivalents to these examples. Accordingly, it is to be understood that the alterations, modification and equivalents of examples of the present patent is to be controlled by the limitations set forth in the claims and any equivalents thereof.

1. A method for co-production of monophenols and cellulose by transition metal oxide catalytic oxidation of biomass, comprising:
(a) loading a reactor with pretreated dry biomass, transition metal oxide and alkali metal hydroxide solution and then the reactor is sealed, evacuated and refilled with O₂, the O₂ pressure being kept at 0.1˜3 MPa, and the mixture is allowed to react at 80˜200° C. for 1˜180 min under stirring, and then cooled down at room temperature to obtain a suspension;
(b) the suspension obtained in step (a) is centrifuged to separate clear supernatant from solid residue, the supernatant being acidified to pH 2˜3, an organic solvent being added to the acidified solution to extract phenolic compounds and the organic phase being gathered; the residual acid and water in the organic solution are removed and monophenols are obtained by vacuum distillation of the organic phase; and
(c) the solid residue obtained in step (b) is placed in a sieve, and the catalyst is rinsed out with water while the biomass residue is retained on the sieve, the aqueous solution containing catalyst is filtered or centrifuged to recycle the spent catalyst, the biomass residue is washed with acid aqueous solution to remove the residual catalyst until the solution is clear, and continuedly washed with water until the pH value of waste liquid reaches 7 and then the biomass residue is dried.

2. The method of claim 1, wherein the dry biomass is lignocellulosic biomass, and wherein the lignocellulosic biomass is selected from a group consisting of hardwood, softwood, grass and agro-forestry residue.

3. The method of claim 1, wherein the pretreatment of dry biomass comprising treatment of the biomass with anhydrous ethanol and refluxing for 8 h, then dried at 50˜60° C.

4. The method of claim 1, wherein the alkali metal hydroxide is selected from a group consisting of NaOH, KOH and Mg(OH)₂.

5. The method of claim 1, wherein the solid-liquid ratio of dry biomass to aqueous solution of hydroxide of alkali metal is 1:20˜50.

6. The method of claim 1, wherein the transition metal oxide is selected from a group consisting of CuO, TiO₂, NiO, Co₂O₃, Fe₂O₃, Fe₃O₄ and ZnO.

7. The method of claim 6, wherein the transition metal oxide is CuO nanoparticles.

8. The method of claim 7, wherein the particle size of CuO nanoparticles is 40˜200 nm.

9. The method of claim 7, wherein the content of CuO nanoparticles is 20˜40% of the weight of the dry biomass.

10. The method of claim 1, wherein the reaction temperature is 120˜180° C.

11. The method of claim 1, wherein the stirring speed is 50˜1000 rpm.

12. The method of claim 1, wherein the reaction time is 10˜120 min.

13. The method of claim 4, wherein the concentration of alkali metal hydroxide aqueous solution is 2.5˜15 wt %.

14. The method of claim 6, wherein the content of transition metal oxide is 1˜100% of the weight of the dry biomass.
